(19) United States
(12) Patent Application Publication
(10) Pub. No.: US 2006/0127325 A1
(43) Pub. Date: Jun. 15, 2006

(54) Dental formulation

(76) Inventor: Dennis James Bannister, Cranebrook , New South Wales (AU)

Correspondence Address: SILLS CUMMIS EPSTEIN & GROSS P.C., ONE RIVERFRONT PLAZA, IP DEPARTMENT, NEWARK, NJ, 07102, US

(21) Appl. No.: 10/540,171

(22) PCT Filed: Dec. 22, 2003
(86) PCT No.: PCT/AU03/01713

§ 371 Date: Dec. 16, 2005

(30) Foreign Application Priority Data
Dec. 20, 2002 (AU) 2002953460

(51) Int. Cl. A61K 8/81; A61C 5/00

(52) U.S. Cl. 424/49; 433/217.1

(57) ABSTRACT

This invention relates to formulations for treating or reducing dentine hypersensitivity that causes pain arising from exposed dentine in the oral cavity. The present invention seeks to provide formulations for physical blockage of dentinal tubules that have long term effectiveness and cause minimal irritation (if any at all) to the tissues of the oral cavity. Preferred formulations should be easy to apply, be immediately effective and maintain dentinal tubule occlusion under normal teeth cleaning regimes that include regular tooth brushing. The invention provides a formulation for reducing dentine sensitivity in the oral cavity, which incorporates at least one physical desensitising agent in form of a light curable monomer that forms a resilient polymer gel upon curing.

### FIELD OF THE INVENTION

This invention relates to formulations for treating or reducing dentine hypersensitivity that causes pain arising from exposed dentine in the oral cavity.

### BACKGROUND OF THE INVENTION

Dentine sensitivity (or hypersensitivity, both terms being used in clinical dental practice interchangeably) is believed to be caused by rapid shifts, in either direction, of the fluids contained within dentinal tubules of teeth, following stimulus application, resulting in activation of sensory nerves in the pulp/inner dentine region of a tooth. The pain producing stimuli can be thermal, tactile, osmotic, chemical or evaporative, but the cold stimulus appears to be the greatest problem. Dentine hypersensitivity is characterized by short, sharp pain arising from the exposed dentine.

Conventional therapies for dentine hypersensitivity are based on using topical applied “desensitising” agents and formulations. These agents can be classified on the basis of their chemical and physical properties, ie there are chemical agents such as corticosteroids, benzalkonium chloride in hydroxy ethylmethacrylate, chlorhexidine, glutaraldehyde, potassium oxalate, fluorides and others that use chemical interaction with the sensory nerves, there are physical agents that act as a barrier to prevent dential tubule fluid contact with stimulus agents, and mixed chemical/physical agents. Physical materials and compounds which have been used for treating or preventing sensitivity in the oral cavity and which aim to block the sensitivity mechanism through tubule occlusion include compounds that interact with proteins of the tooth structure and block the ends of the dentinal tubules, light curable composites and resins, varnishes, glass ionomer cements, sealants and others.

For a more in depth look at the epidemiology, mechanisms of pain reduction and aetiological factors of dentine hypersensitivity reference should be had to the article by R. H. Dababneh, A. T. Khouri and M. Addy, “Dentine hypersensitivity—an enigma? A review of terminology, epidemiology, mechanisms and management, in British Dental Journal, Vol. 187, No. 11, December 1999

Prior art formulations comprising chemically active compounds have the disadvantage that they are generally short lived in effectiveness, and can cause irritation of the soft tissues, such as the gum line. Some active compounds, such as glutaraldehyde, can burn the skin or eyes.

Prior art formulations comprising physical “barrier” compounds are generally hard materials that often exhibit poor adhesion to tooth enamel by themselves and may require dentine bonding agents or surface primers. Also, some of the varnishes and resins employed will be brushed off in time with normal teeth cleaning regimes, resulting in re-opening of the dentinal tubules previously sealed off by the resin.

It is an aim of the present invention to provide formulations for physical blockage of dentinal tubules that have long term effectiveness and cause minimal irritation (if any at all) to the tissues of the oral cavity. Preferred formulations should be easy to apply, be immediately effective and maintain dentinal tubule occlusion under normal teeth cleaning regimes that include regular tooth brushing.

Another aim is to provide a method of treating dentine sensitivity using physical (barrier) formulations that may be brushed (or otherwise applied) onto exposed dentine and provide longer term sensitivity reduction.

### SUMMARY OF THE INVENTION

According to a broad form of the invention there is provided a formulation for reducing dentine sensitivity in the oral cavity, which incorporates at least one physical desensitising agent in form of a light curable monomer that forms a resilient polymer gel upon curing.

According to a further aspect of the invention there is provided a dentine sensitivity reducing formulation that includes a light-cured, form-stable, resilient gel polymer.

A formulation for topical application on dentine may include or be formed from (1) at least one multifunctional polymer or (2) at least one multifunctional polymer and at least one monomer or (3) more than one monomer. A light sensitive polymerisation initiator is mixed with the multifunctional polymer(s) and/or monomer (s) to form a polymer premix in a suitable carrier liquid. This premix can be stored in suitable, light-protected containers, and on demand then applied in the oral cavity to the surface of a painful or sensitive tooth, or to exposed dentine.

The premix and carrier liquid mixture should advantageously have a sufficiently low viscosity to allow fluid migration into exposed dentinal tubules by capillary action, where it forms a liquid plug within the tubule and seals off the tubule opening at the toot surface area.

Light in the wavelength range of 300 to 650 nm, which is the wave length at which most commercially available polymerisation initiators react to initiate polymerisation of suitable, gel-forming monomers employed in the invention, is then directed onto the area to which the mixture has been applied, which causes polymerisation of the monomer into a gel, thereby setting the desensitising polymer formulation within the tubules and forming relatively soft gel plugs that occlude access to the tubules.

The use of light-curing, gel-forming formulations for desensitising teeth has a number of advantages over known methods of physical desensitising. The formulation, once cured, forms a resilient, relatively soft gel plug within the dentinal tubules. The gel plugs, which undergo slight expansion during curing, form a slight resilient press fit within the tubules and thus act as an effective physical barrier to stimulus agents, including liquid ingress from the oral cavity and thermal stimuli. The gel blocks the dentinal tubules and prevents fluid movement within the dentinal tubules. The result is a prevention or reduction of sensitivity or pain. The resiliency and softness of the gel plug avoids creation of strains that are typically encountered with hard setting compounds previously used. Whilst the polymer gel film that is also formed on the tooth surface will eventually be worn away as consequence of tooth brushing, the gel plugs will remain embedded within the tubules, as their resilient nature make them difficult to dislodge and extract from the tubule. Hence, desensitising formulations according to the invention have longer term effectiveness when compared with prior art formulations.

Advantageously, the formulation may include a hydrogel that swells in the presence of moisture in the mouth, causing the gel to resiliently tighten, i.e. form an improved press fit within the tubules compared to the press fit achieved upon curing of the mixture.

The gel polymer is advantageously selected such as to remain permeable to oxygen and electrolytes, just as would be the case with normal tubules.

In another aspect of the invention there is provided a method of preventing or reducing sensitivity or pain in at least one tooth, which method includes applying to said tooth a polymer premix of the invention and curing said premix by application of light.

There is further provided according to the invention the use of at least one multifunctional polymer and/or at least one monomer, together with a light sensitive polymerisation initiator, for the preparation of a formulation for desensitising a tooth.

As employed above and throughout this disclosure (including the claims), the following terms, unless otherwise indicated, shall be understood to have the following meanings:

“Desensitising” is to be taken as meaning reducing sensitivity or pain by physically blocking access to the interior of the dentinal tubules.

“Cured/curing” and grammatical variations thereof refers to the polymerisation process whereby the desensitising polymer plug is formed within the dentinal tubules.

### DETAILED DESCRIPTION OF THE INVENTION

In the following, a description of preferred embodiments of the invention will be provided. Whilst specific chemical compounds are used in the preferred embodiments, it will be appreciated that other polymer compounds may be employed that form a resilient gel body once cured, in contrast with hard curing polymers.

A preferred multifunctional polymer used in a desensitising formulation according to the invention is a polycarboxylic acid polymer. The monomer is preferably an acrylate or allyl derivative. The monomer is selected from hydroxy ethylmethacrylate, glycol dimethacrylate, diallyloxyacetic acid, poly(ethylene glycol) dimethacrylate, 2-acrylamidoglycolic acid, acrylic acid, methacrylic acid, and itaconic acid.

The light sensitive polymerisation initiator is a quinone derivative in combination with a quaternary amine derivative. A preferred formulation uses camphorquinone. The quaternary amine derivative may be selected from N,N,3,5-tetramethyl aniline, poly(ethyleneimine), N,N,N,N-tetraethyldiethylenetriamine, and N,N-diethylethylenediamine, tetramethyl aniline being the most preferred quaternary amine. On application of light in the wavelength range 300 to 650 nm, the light sensitive initiator initiates the polymerisation of the monomer to form the desensitising polymer.

A ready to use formulation is prepared by dissolving the polymer(s) and/or monomer(s) and polymerisation initiator in water, together with a preservative (to improve shelf life). A typically used preservative is butylated hydroxy toluene, although other preservatives such as hydroquinone, and methyl hydroquinone may be used.

The relative amounts of the constituents of the formulation may vary within certain ranges. Broadly, the constituents may be present as follows:

| | |
|---|---|
| Polycarboxylic acid polymer | about 1 to about 50% by weight |
| 2-Hydroxy ethylmethacrylate | about 10 to about 80% by weight |
| Glycol dimethacrylate | about 1 to about 50% by weight |
| Water | about 1 to about 70% by weight |
| Camphorquinone | about 0.01 to about 5% by weight |
| Tetramethyl aniline | about 0.01 to about 5% by weight |
| Butylated hydroxy toluene | about 0.01 to about 5% by weight |

Test batches of formulations in accordance with the invention have shown good tubule occlusion results where constituents were present in the formulation within the following ranges:

| | |
|---|---|
| Polycarboxylic acid polymer | about 5 to about 15% by weight |
| 2-Hydroxy ethylmethacrylate | about 50 to about 80% by weight |
| Glycol dimethacrylate | about 3 to about 9% by weight |
| Water | about 5 to about 25% by weight |
| Camphorquinone | about 0.1 to about 1% by weight |
| Tetramethyl aniline | about 0.1 to about 1% by weight |
| Butylated hydroxy toluene | about 0.01 to about 0.1% byweight |

Excellent results in creating a soft, resilient gel after curing have been obtained with a formulation that comprises

| | |
|---|---|
| Polycarboxylic acid polymer | about 7.5% by weight |
| 2-Hydroxy ethylmethacrylate | about 74.5% by weight |
| Diallyloxyacetic acid, sodium salt | about 6% by weight |
| Water | about 12% by weight |
| Camphorquinone | about 0.2% by weight |
| Tetramethyl aniline | about 0.22% by weight |
| Butylated hydroxy toluene | about 0.05% by weight |

In the above formulations, water could be replaced by another, suitable solvent that provides the initial swelling agent for the formulation; after curing into the soft, resilient gel plug, the solvent will diffuse out of the gel at a slow rate and saliva will then concurrently provide the moisture that is necessary to replace the solvent as swelling agent.

A desensitising hydrogel formulation in accordance with the invention was prepared as follows:

Ingredients

| | |
|---|---|
| Gantrez AN119BF | 7.5 grams |
| Deionised water | 12.0 grams |
| HEMA | 74.5 grams |
| Diallyloxyacetic acid, sodium salt | 6.0 grams |
| Camphorquinone | 0.20 grams |
| BHT | 0.05 grams |
| Tetramethyl aniline | 0.22 grams |

Gantrez AN119BF is an alternating copolymer of vinyl methyl ether and maleic anhydride, HEMA is 2-hydroxy ethyl methacrylate and BHT is butylated hydroxy toluene.

Method

The equipment requirements were as follows:

a) Balance weighing in grams and reading to two decimal places

b) Pasteur pipettes

c) Two clean 500 ml beakers

d) Plastic spatula

e) Ultrasonic bath

A clean glass beaker was placed on the balance and the balance tared. To the glass beaker was added 7.5 grams of Gantrez AN119BF, 12.0 grams of deionised water, 74.5 grams of HEMA, and 6.0 grams of diallyloxyacetic acid sodium salt. The mixture was stirred with a plastic spatula and then the beaker covered with plastic film. Water was poured into the ultrasonic bath to a depth of about one centimeter, and then the beaker placed in the ultrasonic bath. The ultrasonic bath was turned on to agitate the contents of the beaker until the mixture became clear, and there were no gel particles.

In an orange light production area, 0.20 grams of camphorquinone, 0.05 grams of BHT, and 0.22 grams of tetramethyl aniline was weighed into a clean glass beaker. Then the solution containing the Gantrez AN119BF was added to this beaker. The beaker was covered with plastic film and agitated in the ultrasonic bath until all the camphorquinone and BHT had dissolved.

The resultant product (the “premix”) was packaged, ready for delivery.

This premix can then be applied to a painful or sensitive tooth, or to exposed dentine. Naturally, the premix can also be applied to a tooth which has no pain, but in anticipation of pain, for example before a surgical procedure. Light is directed onto the area to which the premix is applied to cause polymerisation. The desensitising polymer so formed reduces or prevents pain.

1. A formulation for reducing dentine sensitivity in the oral cavity, which incorporates at least one physical desensitizing agent in form of a light curable monomer that forms a resilient polymer gel upon curing.

2. A dentine sensitivity reducing formulation that includes a light-cured, form-stable, resilient gel polymer.

3. The formulation of claim 1, including a light sensitive polymerization initiator and (1) at least one multifunctional polymer, or (2) at least one multifunctional polymer and at least one monomer, or (3) more than one monomer.

4. The formulation of claim 3 in a suitable carrier liquid.

5. The formulation of claim 4, wherein the carrier liquid includes water.

6. The formulation of claim 4 having a viscosity to allow fluid migration into exposed dentinal tubules by capillary action.

7. The formulation claim 1, including a gel polymer that swells in the presence of moisture.

8. The formulation of an claim 7, wherein the gel polymer is permeable to oxygen and electrolytes.

9. The formulation of claim 3, including a polycarboxylic acid polymer.

10. The formulation of claim 3, including an acrylate or allyl derivative.

11. The formulation of claim 3, wherein the monomer is selected from the group consisting of 2-hydroxy ethylmethacrylate, glycol dimethacrylate, diallyloxyacetic acid, poly(ethylene glycol) dimethacrylate, 2-acrylamidoglycolic acid, acrylic acid, methacrylic acid, and itaconic acid.

12. The formulation of claim 3, wherein the light sensitive polymerization initiator is a quinone derivative in combination with a quaternary amine derivative.

13. The formulation of claim 12, incorporating camphorquinone and a quaternary amine derivative selected from the group consisting of N,N,3,5 tetramethyl aniline, poly(ethyleneimine), N,N,N,N-tetraethyidiethylenetriamine, and N,N-diethylethylenediamine.

14. The formulation of claim 3, further including a preservative such as butylated hydroxy toluene or hydroquinone, in particular methyl hydroquinone.

15. The formulation of claim 3, having the following constituents in % values by weight: Polycarboxylic acid polymer about 1 to about 50, 2-hydroxy ethylmethacrylate about 10 to about 80, Glycol dimethacrylate about 1 to about 50, Water about 1 to about 70, Camphorquinone about 0.01 to about 5, Tetramethyl aniline about 0.01 to about 5, and Butylated hydroxy toluene about 0.01 to about 5.

16. The formulation of claim 15, wherein the constituents are present in the following amount in % by weight: Polycarboxylic acid polymer about 5 to about 15, 2-hydroxy ethylmethacrylate about 50 to about 80, Glycol dimethacrylate 3 of 5 about 3 to about 9, Water about 5 to about 25, Camphorquinone about 0.1 to about 1, Tetramethyl aniline about 0.1 to about 1 and Butylated hydroxy toluene about 0.01 to about 0.1.

17. The formulation of claim 16, wherein the constituents are present in the following amounts: 1 0 Polycarboxylic acid polymer about 7.5% by weight 2-Hydroxy ethylmethacrylate about 74.5% by weight Diallyloxyacetic acid, sodium salt about 6% by weight Water about 12% by weight Camphorquinone about 0.2% by weight Tetramethyl aniline about 0.22% by weight Butylated hydroxy toluene about 0.05% by weight

18. A method of preventing or reducing sensitivity or pain in teeth, which method includes applying a formulation in accordance with claim 1 to exposed dentinal tubules of teeth, allowing said formulation to migrate into the tubules, and curing the formulation by application of light with a wave length in the range of 300 to 650 nm, whereby soft resilient gel plugs are formed within the tubules.
